SEQUENCE LISTING (14 sequences)

SEQ ID NO: 1 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 2 - protein, 61 aa, Homo sapiens
MKRFLFLLLTISLLVMVQIQTGLSGQNDTSQTSSPSASSNISGGIFLFFVANAIIHLFCFS

SEQ ID NO: 3 - protein, 359 aa, Homo sapiens
MAEGTLWQILCVSSDAQPQTFEGVKGADPPTLPPGSFLPGPVLWWGSLARLQTEKSDEVSRKGNWWVTEMGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 4 - protein, 233 aa, Homo sapiens
MWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 5 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 6 - protein, 310 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 7 - protein, 323 aa, Homo sapiens
MGILSFLPVLATESDWADCKSPQPWGHMLLWTAVLFLAPVAGTPAAPPKAVLKLEPQWINVLQEDSVTLTCRGTHSPESDSIPWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANSTDPVKAAQFEPPGRQMIAIRKRQPEETNNDYETADGGYMTLNPRAPTDDDKNIYLTLPPNDHVNSNN

SEQ ID NO: 8 - protein, 119 aa, Homo sapiens [sequence omitted]

SEQ ID NO: 9 - protein, 160 aa, Homo sapiens
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKP

SEQ ID NO: 10 - protein, 293 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 11 - protein, 229 aa, Artificial Sequence
  Description of Artificial Sequence Synthetic polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSV

SEQ ID NO: 12 - protein, 240 aa, Artificial Sequence
  Description of Artificial Sequence Synthetic polypeptide
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIY

SEQ ID NO: 13 - protein, 313 aa, Artificial Sequence
  Description of Artificial Sequence Synthetic polypeptide
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTI

SEQ ID NO: 14 - protein, 404 aa, Mus sp.
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVKIHRLQREKKYNLEVPLVSEQGKKANSFQQVRSDGVYEEVTATASQTTPKEAPDGPRSSVGDCGPEQPEPLPPSDSTGAQTSQS

(19) United States
(12) Patent Application Publication
(10) Pub. No.: US 2024/0139341 A1
(43) Pub. Date: May 2, 2024

(54) LENTIVIRUS PROTECTION VIA Fc OVEREXPRESSION

(71) Applicant: THE REGENTS OF THE UNIVERSITY OF CALIFORNIA, Oakland, CA (US)

(72) Inventor: Tobias Deuse, Burlingame, CA (US)

(73) Assignee: THE REGENTS OF THE UNIVERSITY OF CALIFORNIA, Oakland, CA (US)

(21) Appl. No.: 18/279,199

(22) PCT Filed: Mar. 7, 2022
(86) PCT No.: PCT/US2022/019203

§ 371 Date: Aug. 28, 2023

Related U.S. Application Data
(60) Provisional application No. 63/158,204, filed on Mar. 8, 2021.

(51) Int. Cl. A61K 48/00; C12N 15/86

(52) U.S. Cl. CPC A61K 48/0041; C12N 15/86; C12N 2740/15043; C12N 2740/15052

(57) ABSTRACT

The invention relates to gene therapies using viral vectors. The invention provides, for the first time, packaging cells that comprise enhanced Fc receptor expression (e.g. CD16, CD32, or CD64) to generate gene therapy viruses that evade antibody-dependent inactivation (ADI) or complement-dependent inactivation (CDI). The invention also provides gene therapy viruses with the enhanced Fc receptors displayed on their viral envelope.

### CROSS REFERENCE TO RELATED APPLICATIONS

This application claims priority under 35 U.S.C. § 119(e) to U.S. Provisional Application No. 63/158,204, filed Mar. 8, 2021, and is incorporated herein by reference in their entirety

### FIELD OF THE INVENTION

The invention relates to gene therapies using viral vectors. The invention provides, for the first time, packaging cells that comprise enhanced Fc receptor expression (e.g. CD16, CD32, or CD64) to generate gene therapy viruses that evade antibody-dependent cellular inactivation (ADI) by immune cells or complement-dependent inactivation (CDI). The invention also provides gene therapy viruses with the enhanced Fc receptors displayed on their viral envelope.

The invention provides gene therapies that comprises administering to a subject the gene therapy viruses described herein comprising a genetic element for insertion into the patient's genome. In some embodiments, the gene therapy virus is a lentivirus. In other embodiments, the cell-surface receptors evade viral opsonization by Fc sequestration. In other embodiments, the gene therapy virus is produced in packaging cells with an elevelated Fc receptor expression. In other embodiments, the genetic element is a gene for expression in cells within said subject.

### BACKGROUND

The invention provides, for the first time, gene therapy viruses and packaging cell lines for producing them that comprise enhanced Fc receptor (e.g. CD16, CD32, or CD64) expression. These receptors sequester antibodies to increase the gene therapy virus effectiveness.

Gene therapy has the potential to correct genetic diseases by introducing genes that are abnormal, missing or deficient. It may also be used to make beneficial proteins. Gene therapies introduce genetic materials into cells. Gene delivery should ideally target specific cell types, have no off-target effects, and result in lasting gene expression.

Gene therapies typically use viral vectors that are genetically engineered to deliver genes (“gene therapy viruses”). The viruses are modified to avoid diseases when used in people. Gene therapy viruses can be injected directly into a specific tissue in the body where it is taken up by individual cells, or given intravenously (IV). If the treatment is successful, the new gene delivered by the vector may make a functioning protein.

### SUMMARY OF THE INVENTION

The invention provides, for the first time, packaging cells that comprise enhanced Fc receptor expression on the cell surface (e.g. CD16, CD32, CD64, or intracellular signalling domain truncations thereof) to produce gene therapy viruses incorporating the cell-surface receptors in their envelope membranes. In some embodiments, the Fc receptors help the gene therapy viruses evade viral opsonization, ADI, or CDI by a mechanism of Fc sequestration. In other embodiments, the Fc receptors help protect the viruses from complement-dependent inactivation (CDI) mechanisms. In other embodiments, the overexpression of either one of the Fcγreceptors I-IV, or a combination of those, imparts enhanced protection.

The invention thus provides that expressing an Fc receptor such as the CD16, CD32, CD64 receptor, or intracellular signalling domain truncations thereof, on viral envelopes sequesters the Fc portion of local antibodies and thus inhibits opsonization, ADI, or CDI. CD64 is constitutively found only on macrophages and monocytes. It is more commonly known as Fc-gamma receptor 1 (FcγRI) and binds IgG Fc regions with high affinity. CD64 expression on gene therapy viruses sequesters the IgG Fc and binds it to the virus.

Thus, the invention provides a gene therapy virus, comprising a transgenic genetic element and a viral envelop having a membrane-bound Fc Receptor protein. In some aspects, the membrane-bound protein is CD16, CD32, CD64, or truncated CD64 (CD64t). In a preferred aspect, the membrane-bound protein is CD64. In another preferred aspect, the membrane-bound protein is CD32.

The invention provides a gene therapy virus as disclosed herein, wherein the virus is a lentivirus, retrovirus, or a sendai virus.

The invention provides a modified packaging cell, wherein the modified cell has an elevated level of an Fc receptor protein expression when compared to a parental version of the modified packaging cell, wherein a first gene therapy virus produced in the modified packaging cell resists opsonization, complement-dependent inactivation (CDI) or antibody-dependent inactivation (ADI) by immune cells in a subject when compared to a second gene therapy virus produced in the parental version of the modified packaging cell. In some aspects, the elevated Fc receptor protein is CD16, CD32, or CD64. In a preferred aspect, the elevated Fc receptor protein is CD64 and in other aspects has at least a 90% sequence identity to SEQ ID NO:1. In a more preferred aspect, the CD64 protein has the sequence of SEQ ID NO:1.

In some aspects of the invention, the modified packaging cell is from a species that is selected from the group consisting of a human, monkey, cow, pig, chicken, mouse, rat, hamster, guinea pig, and insect. In other aspects, the modified packaging cell is derived from a human HEK293T cell, a LentiPro26 cell, a STAR cell, or an RD2-MolPack-Chim3 cell. In a preferred aspect, the modified packaging cell is derived from a HEK293T cell.

In other aspects of the invention, the increased CD16, CD32, CD64, or truncated CD64 (CD64t) expression results from introducing at least one copy of a human CD16, CD32, CD64, or truncated CD64 (CD64t) gene under the control of a promoter into the parental version of the modified pluripotent cell. In other apsects, the promoter is a constitutive promoter.

The invention provides a modified packaging cell or a cell derived therefrom as disclosed herein, further comprising a reduced HLA I expression level. In some aspects, the HLA I function is reduced by a reduction in expression of a β-2 microglobulin, HLA-A, HLA-B, or HLA-C protein. In preferred aspects, the β-2 microglobulin, HLA-A, HLA-B, or HLA-C protein expression is eliminated. In other aspects, a gene encoding the β-2 microglobulin, HLA-A, HLA-B, or HLA-C protein is knocked out.

The the invention provides a modified packaging cell as disclosed herein further comprising a reduced HLA II expression level. In some aspects, the HLA II function is reduced by a reduction in expression of a RFXS, RFXANK, RFXAP, or CIITA, HLA-DP, HLA-DR, or HLA-DQ protein. In preferred aspects, the RFXS, RFXANK, RFXAP, or CIITA, HLA-DP, HLA-DR, or HLA-DQ protein expression is eliminated. In other apsects, a gene encoding the RFXS, RFXANK, RFXAP, or CIITA, HLA-DP, HLA-DR, or an HLA-DQ protein is knocked out.

The invention provides a modified packaging cell as disclosed herein, further comprising an increased CD47 expression when compared to a parental packaing cell. In some aspects, the increased CD47 protein expression results from a modification to an endogenous CD47 gene locus. In other aspects, the increased CD47 protein expression results from a CD47 transgene.

The invention provides a modified packaging cell as disclosed herein, wherein the cell is ABO blood group type O. In some aspects, the cell has a reduced or eliminated ABO blood group antigen selected from the group consisting of A1, A2, and B.

The invention provides a modified packaging cell as disclosed herein, wherein the the cell is Rhesus factor negative (Rh-). In some aspects, the cell has a reduced or eliminated Rh protein antigen expression selected from the group consisting of Rh C antigen, Rh E antigen, Kell K antigen (KEL), Duffy (FY) Fya antigen, Duffy Fy3 antigen, Kidd (JK) Jkb antigen, MNS antigen U, and MNS antigen S.

The invention provides a gene therapy virus comprising a therapeutic transgenic genetic element and a viral envelope, wherein the gene therapy virus is produced from the modified packaging cells disclosed herein. In some aspects, the viral envelop has no HLA I proteins, no HLA I proteins, or comprises CD47 proteins. In other aspects, the gene therapy virus has an ABO blood type O phenotype or an Rh(−) phenotype.

The invention provides a pharmaceutical composition for treating a disease in a subject, comprising a gene therapy virus as disclosed herein and a pharmaceutically acceptable carrier. The invention provides a medicament, comprising a gene therapy virus as disclosed herein and a pharmaceutically acceptable carrier.

The invention provides a gene therapy virus as disclosed herein for treating a disease in a subject.

The invention provides a method of manufacturing a pharmaceutical composition, comprising combining the gene therapy virus as disclosed herein with a pharmaceutical carrier.

The invention provides a method of treating a genetic disease, comprising administering a gene therapy virus as disclosed herein to a subject. In some aspects, the genetic disease is selected from the group consisting of X-linked severe combined immunodeficiency (X-SCID), Stargardt Disease, Usher Syndrome, Choroideremia, Achromatopsia, X-linked Retinoschisis, b-thalassemia, Sickle Cell Disease, Hemophilia, Wiskott-Aldrich Syndrome, X-linked Chronic Granulomatos Disease, Mucopolysaccharidosis IIIB, Aromatic L-Amino Acid Decarboxylase Deficiency, Recessive Dystrophic Epidermolysis Bullosa, Mucopolysaccharidosis Type I (Hurler Syndrome), Alpha 1 Atnitrypsin Deficiency, Homozygous Familial Hypercholesterolemia, Hutchinson-Gilford Progeria Syndrome (HGPS), Acondroplasia, MECP2 Duplication Syndrome, Pendred Syndrome, Leber Hereditary Optic Neuropathy, Noonan Syndrome, Congenital Myasthenic Syndrome, and Hereditary Hemorhagic Telangiectasia.

The invention provides a use of a gene therapy virus as disclosed herein for preparing a therapeutic composition for treating a genetic disease.

The invention provides a use of a gene therapy virus as disclosed herein for treating a genetic disease.

The invention provides a medicament for treating a genetic disease comprising a gene therapy virus as disclosed herein.

### BRIEF DESCRIPTION OF THE DRAWINGS

FIG. 1 exemplifies CDI of a lentivirus that has budded from a packaging cell. Packaging cell human leukocyte antigen (HLA) molecules, other cell membrane proteins, and viral proteins are on the lentiviral envelope. Upon the first dose of a gene therapy virus, the patient becomes immunized against the viral and packaging cell proteins. When the lentivirus is re-dosed, patient IgG antibodies bind viral particles and induce complement-dependent lysis of the virus. This inactivates the virus and limits its therapeutic efficacy.

FIG. 2 shows a packaging cell expressing an FcR (e.g. CD64). When incorporated into the viral envelope, the virus captures IgG antibody by their Fc and is protected from the antibody binding via the Fab. This prevents opsonization, ADI, or CDI upon redosing.

FIG. 3A and 3B shows the expression of CD64 on packaging cells. FIG. 3A shows a flow cytometry histogram of 293t cells. These cells do not express CD64. FIG. 3B shows a flow cytometry histogram of 293t (CD64) cells, which have strong CD64 expression.

FIGS. 4A and 4B shows a packaging cell membrane expressing CD64 capturing free IgG Fc. FIG. 4A shows a flow cytometry histogram of IgG Fc bound to the parental 293t cells. The 293t cells (negative for CD52) were exposed to increasing concentrations of an anti-CD52 antibody IgG1 with human Fc (alemtuzumab). There was no measurable IgG Fc binding. FIG. 4B shows that alemtuzumab was bound to the 293t cells expressing CD64 via Fc in a concentration-dependent manner. This shows that CD64 captured and bound free IgG via its Fc domain.

FIG. 5 shows 293t and 293t (CD64) plated on the XCelligence platform for in vitro impedance assays. Both 293t and 293t (CD64) cells express the HLA-A2 antigen. A humanized anti-HLA-A2 IgG1 antibody was capable of mediating complement-dependent cytotoxicity (CDC). The upper row shows that increasing concentrations of anti-HLA-A2 led to increasing efficacy of 293t killing via CDC. The lower row shows that 293t (CD64) were protected against anti-HLA-A2-mediated CDC over several orders of magnitude.

FIG. 6A and 6B shows the expression of mouse Fcgr1 on packaging cells. FIG. 6A shows a flow cytometry histogram of 293t cells. These cells do not express Fcgr1. FIG. 6B shows a flow cytometry histogram of 293t-Fcgr1 cells, which have strong Fcgr1 expression.

FIGS. 7A and 7B shows a packaging cell membrane expressing Fcgr1 capturing free mouse IgG2a Fc. FIG. 7A shows a flow cytometry histogram of anti-CD20 mouse IgG2a Fc bound to the parental 293t cells. The 293t cells (negative for CD20) were exposed to increasing concentrations of an anti-CD20 mouse antibody IgG2a. There was no measurable IgG2a Fc binding. FIG. 7B shows that anti-CD20 mouse antibody IgG2a was bound to the 293t cells expressing Fcgr1 via Fc in a concentration-dependent manner. This shows that Fcgr1 captured and bound free IgG2a via its Fc domain.

FIGS. 8A and 8B show Enzyme Linked Immunosorbant (ELISA) assays for the binding of antibodies to lentiviruses. ELISA plates were coated with 293t or 293t-Fcgr1 lentiviruses. FIG. 8A shows similar binding of anti-VSV-G, suggesting that the pseudotyping with VSV-G was not different among lentiviruses. FIG. 8B shows significantly stronger binding of anti-Fcgr1 antibody to 293t lentiviruses suggesting increased expression of Fcgr1 on the envelope.

FIG. 9 shows an ELISA assay with coated lentivirus particles and binding of anti-CD20 mouse IgG2a antibody over a concentration rage. Lentiviruses do not express CD20, so the binding must occur via Fc and Fcgr1. While 293t lentiviruses did not show any antibody binding, 293t-Fcgr1 showed concentration-dependent binding.

FIG. 10A and 10B shows in vitro lentivirus inactivation assays. Mouse endothelial cells were plated and lentiviruses expressing firefly luciferase were added. In addition, mouse serum and increasing concentrations of an anti-VSV-G mouse IgG2b antibody were added. The antibody with serum can cause CDI before the lentivirus can deliver its payload. In FIG. 10A, 293t lentivirus was used. In a concentration-dependent manner and starting at low concentrations, lentiviruses were inactivated before transducing the target cells, which resulted in very low bioluminescence emission. In FIG. 10B, 293t-Fcgr1 lentivirus was used. There was a much smaller effect from anti-VSV-G antibodies on the efficacy of firefly transduction and target cells showed bioluminescence in all but the highest antibody concentration.

FIG. 11 shows an in vitro lentivirus inactivation assay. BALB/c mice were intravenously injected with 30 million lentiviral particles packaged in 293t cells and 28 days later, serum containing anti-lentiviral antibodies was drawn. Target human endothelial cells were plated in 96 wells coated with gelatine and allowed to attach overnight. The next day, 2 μl lentivirus 293t or 293t (CD64) was incubated with 50 μl serum containing anti-lentiviral antibodies. Then the serum/lentivirus mix was added to the endothelial cells and 2 days later, the transduction efficacy is measured using BLI imaging. The lentivirus packaged in 293t (CD64) achieve higher BLI signals (FIG. 11).

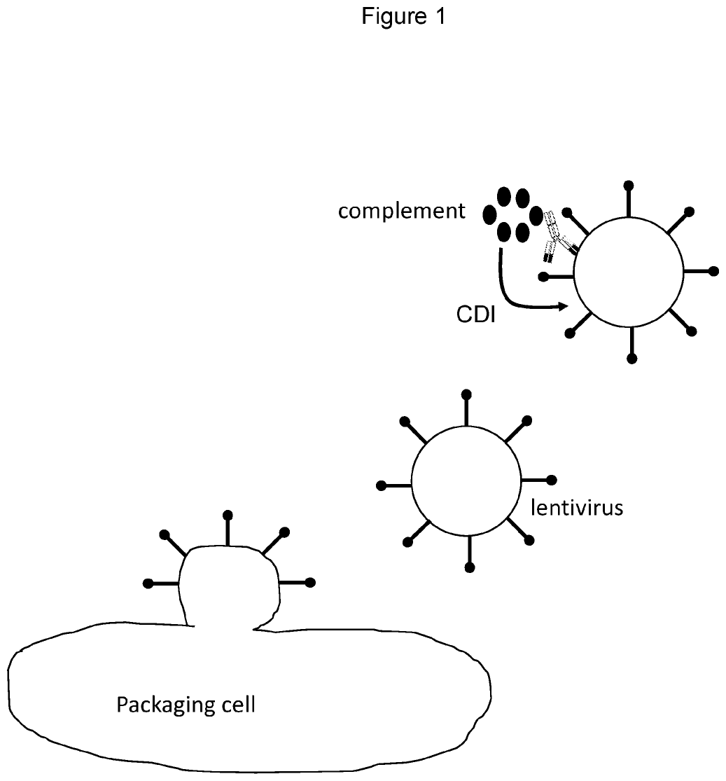

### DETAILED DESCRIPTION

The invention provides, for the first time, gene therapy viruses and packaging cell lines for producing them that comprise enhanced Fc receptor expression (e.g. CD16, CD32, CD64 or intracellular signalling domain truncations thereof). These receptors sequester antibodies to increase the gene therapy virus effectiveness. Upon a first dose of a gene therapy virus, the subject receiving the virus may generate an immune response to it. Some subjects even have virus-binding antibodies before their first application. By incorporating the Fc receptors, upon re-dosing, IgG is sequestered by their Fc portion. The Fc domains are unavailable to bind and activate complement or activate other immune responses such as engulfment, opsonization, ADI, or CDI.

Gene therapy has the potential to correct genetic diseases by introducing genes that are abnormal, missing or deficient. It may also be used to make beneficial proteins. Gene therapy introduces genetic material into cells. Gene delivery should ideally target specific cell types, have no off-target effects, and result in lasting gene expression.

Gene therapies typically use viral vectors that are genetically engineered to deliver genes (“gene therapy viruses”). The viruses are modified so they can't cause disease when used in people. Gene therapy viruses can be injected directly into a specific tissue in the body where it is taken up by individual cells or given intravenously (by IV). Alternately, a sample of the patient's cells can be removed and exposed to the vector in a laboratory setting. The cells containing the vector are then returned to the patient. If the treatment is successful, the new gene delivered by the vector will make a functioning protein.

One type of gene therapy virus are retroviruses. The genetic material in retroviruses is in the form of RNA molecules, while the genetic material of their hosts is in the form of DNA. When a retrovirus infects a host cell, it will introduce into the cell its RNA together with reverse transcriptase and integrase. The RNA is reverse transcribed before integrating into the genetic material of the host cell genome. An example of a retroviral gene therapy is being tested to treat X-linked severe combined immunodeficiency (X-SCID).

A common retroviral virus vector used in gene therapy trials is the lentivirus Simian immunodeficiency virus coated with an envelope protein, G-protein from Vesicular stomatitis virus. This vector is referred to as VSV G-pseudotyped lentivirus. It infects an almost universal set of cells.

Lentiviruses are currently being explored because they are efficient, incorporate their genetic sequence into the genome, and can be engineered to target specific cells. Lentiviruses, however, are subject to antibody-mediated mediated defenses mounted by the recipients. Antibodies neutralize viruses, rendering them incapable of infecting host cells. They can also cause virus particles to stick together in a process called agglutination. Agglutinated viruses make an easier target for immune cells than single viral particles. Antibodies can also activate phagocytes. Virus-bound antibodies bind to Fc receptors on the surface of phagocytic cells and trigger phagocytosis. Virus-bound antibodies can also bind to Fc receptors on NK cells and induce virus inactivation. Finally, antibody Fc domains can also activate the complement system and lead to virus inactivation. Together, antibodies opsonize viruses, activate the complement system, and promote inactivation or phagocytosis. Complement can also damage the envelope (phospholipid bilayer) that is present on some types of viruses. This antibody barrier makes re-dosing of gene therapy virus particles practically impossible and limits lentiviral-based gene therapy to one dose.

Gene therapy vectors can be sensitive to the mechanisms of B-cells or granulocytes and complement-dependent inactivation (CDI) via activation of the complement cascade. They utilize antibodies that bind to virus and activate the effector immune cells or complement. IgG antibodies can be potent mediations of CDI. They have two variable Fab regions which bind to specific epitopes. The crystalizable Fc region sticks out and serves for the binding of NK cells, B-cells, macrophages, granulocytes, or complement.

Receptors that recognize the Fc portion of IgG are divided into four different classes: FcγRI (CD64), FcγRII (CD32), FcγRIII (CD16), and FcγRIV. Whereas FcγRI displays high affinity for the antibody constant region and restricted isotype specificity, FcγRII and FcγRIII have low affinity for the Fc region of IgG but a broader isotype binding pattern, and FcγRIV is a recently identified receptor with intermediate affinity and restricted subclass specificity. Physiologically, FcγRI functions during early immune responses, while FcγRII and RIII recognize IgG as aggregates surrounding multivalent antigens during late immune responses.

If an antibody binds to an unprotected virus via its Fab regions, the Fc can be bound by complement and activate its cascade to form the membrane attack complex (MAC) for CDI. This antibody barrier makes re-dosing of lentiviral particles practically impossible and limits lentiviral-based gene therapy to one dose.

Because lentiviruses incorporate the packaging cell membranes into their viral envelope, the invention provides packaging cell lines with high Fc receptor expression levels. The invention further provides lentiviral particles with high Fc receptor levels in the viral envelope. These novel lentiviruses absorb IgG antibodies via their Fc receptors and prevent the Fc portions of the antibodies from activating complement, other immune cells, antibody-based viral immunity, or neutralization. This averts antibody-mediated clearance of gene therapy virus particles and increases the efficacy of re-dosing.

In humans, CD16 comes as CD16A and CD16B. They have a 96% sequence similarity in the extracellular immunoglobulin binding regions (aka. FCGR3A and FCGR3B). For FCGR3A, there are several isoforms and there is no clear principal isoform.

In humans, there are three major CD32 subtypes, namely CD32A, CD32B, and CD32C. While CD32A and CD32C are involved in activating cellular responses, CD32B is inhibitory.

Beyond retroviruses, other DNA viruses (Herpesviruses, Poxviruses, Hepadnaviruses, and Asfarviridae) or RNA viruses (Flaviviruses, Alphaviruses, Togaviruses, Coronaviruses, Hepatitis D, Orthomyxoviruses, Paramyxoviruses, Rhabdovirus, Bunyaviruses, and Filoviruses) are enveloped viruses and could show Fc receptor expression. In particular embodments, Sendai viruses are enveloped and engineered for Fc receptor expression.

Another type of gene therapy vector are adenoviruses or adeno-associated viruses. They are double-stranded DNA viruses that cause respiratory, intestinal, and eye infections in humans, including the common cold. Adenoviruses introduce their DNA into the host but is not incorporated into the host cell's genetic material. They are transient and do not replicate. The DNA molecules are transcribed just like any other gene. Because the adenoviral DNA is not replicated, it is not transferred to descendants of infected cells.

Sendai viruses are also used for gene therapy. They naturally replicate in respiratory epithelial cells and are a major pathogen causing respiratory symptoms in mice. In addition to the lung and airway epithelium, recombinant sendai virus vectors can induce strong transgene expression in the cardiovascular system, retinal epithelium, hepatocytes, colonic epithelium, neurons, dendritic cells, and in human hematopoietic stem cells. The application of sendai virus vectors in gene therapy is dependent on, and is on some occasions restricted by, powerful but transient gene expression, wide host cell specificity, low pathogenicity, and strong immunogenicity. To date, sendai virus vectors are being tested clinically for critical limb ischemia and in cancer gene therapy.

As used herein, the terms “subject” or “patient” refers to any animal, such as a domesticated animal, zoo animal, mouse, rat, hamster, guinea pig, primate, or a human. The “subject” or “patient” can be a mammal like a dog, cat, bird, livestock, or a human. Specific examples of “subjects” and “patients” include, but are not limited to, individuals (particularly human) with a disease or disorder related to the liver, heart, lung, kidney, pancreas, brain, neural tissue, blood, bone, bone marrow, and the like.

Mammalian cells can be from humans or non-human mammals. Exemplary non-human mammals include, but are not limited to, mice, rats, cats, dogs, rabbits, guinea pigs, hamsters, sheep, pigs, horses, bovines, and non-human primates (e.g., chimpanzees, macaques, and apes).

By “gene knock out” herein is meant a process that renders a particular gene inactive in the host cell in which it resides, resulting either in no protein of interest being produced or an inactive form. As will be appreciated by those in the art and further described below, this can be accomplished in a number of different ways, including removing nucleic acid sequences from a gene, or interrupting the sequence with other sequences, altering the reading frame, or altering the regulatory components of the nucleic acid. For example, all or part of a coding region of the gene of interest can be removed or replaced with “nonsense” sequences, all or part of a regulatory sequence such as a promoter can be removed or replaced, translation initiation sequences can be removed or replaced, etc.

By “gene knock in” herein is meant a process that adds a genetic function to a host cell. This causes increased levels of the encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

By “wild type” refers to a virus, cell, organism, microorganism, or gene found in nature.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

“Inhibitors,” “activators,” and “modulators” affect a function or expression of a biologically-relevant molecule. The term “modulator” includes both inhibitors and activators. They may be identified using in vitro and in vivo assays for expression or activity of a target molecule.

“Inhibitors” are agents that, e.g., inhibit expression or bind to target molecules or proteins. They may partially or totally block stimulation or have protease inhibitor activity. They may reduce, decrease, prevent, or delay activation, including inactivation, desensitizion, or down regulation of the activity of the described target protein. Modulators may be antagonists of the target molecule or protein.

“Activators” are agents that, e.g., induce or activate the function or expression of a target molecule or protein. They may bind to, stimulate, increase, open, activate, or facilitate the target molecule activity. Activators may be agonists of the target molecule or protein.

“Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a specific embodiment, homologous or derivative sequences share at least an 80 or 85 percent sequence identity. In a specific embodiment, homologous or derivative sequences share at least a 90 percent sequence identity. In a specific embodiment, homologous or derivative sequences share at least a 95 percent sequence identity. In a more specific embodiment, homologous or derivative sequences share at least an 50, 55, 60, 65, 70, 75, 85, 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

“Hybridization” generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al, Current Protocols in Molecular Biology, Wiley Interscience Publishers (1995), incorporated by reference herein in its entirety.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures.

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 Mm sodium phosphate buffer at Ph 6.5 with 750 Mm sodium chloride, 75 Mm sodium citrate at 42° C.; or (3) overnight hybridization in a solution that employs 50% formamide, 5×SSC (0.75 M NaCl, 0.075 M sodium citrate), 50 Mm sodium phosphate (Ph 6.8), 0.1% sodium pyrophosphate, 5× Denhardt's solution, sonicated salmon sperm DNA (50 μl/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein the term “modification” refers to an alteration that physically differentiates the modified molecule from the parent molecule. In one embodiment, an amino acid change in a variant polypeptide prepared according to the methods described herein differentiates it from the corresponding parent that has not been modified according to the methods described herein, such as wild-type proteins, a naturally occurring mutant proteins or another engineered protein that does not include the modifications of such variant polypeptide. In another embodiment, a variant polypeptide includes one or more modifications that differentiates the function of the variant polypeptide from the unmodified polypeptide. For example, an amino acid change in a variant polypeptide affects its receptor binding profile. In other embodiments, a variant polypeptide comprises substitution, deletion, or insertion modifications, or combinations thereof. In another embodiment, a variant polypeptide includes one or more modifications that increases its affinity for a receptor compared to the affinity of the unmodified polypeptide.

In one embodiment, a variant polypeptide includes one or more substitutions, insertions, or deletions relative to a corresponding native or parent sequence. In certain embodiments, a variant polypeptide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31-40, 41 to 50, or 51 or more modifications.

By “episomal vector” herein is meant a genetic vector that can exist and replicate autonomously in the cytoplasm of a cell; e.g. it is not integrated into the genomic DNA of the host cell. A number of episomal vectors are known in the art and described below.

By “knock out” in the context of a gene means that the host cell harboring the knock out does not produce a functional protein product of the gene. As outlined herein, a knock out can result in a variety of ways, from removing all or part of the coding sequence, introducing frameshift mutations such that a functional protein is not produced (either truncated or nonsense sequence), removing or altering a regulatory component (e.g. a promoter) such that the gene is not transcribed, preventing translation through binding to mRNA, etc. Generally, the knockout is effected at the genomic DNA level, such that the cells' offspring also carry the knockout permanently.

By “knock in” in the context of a gene means that the host cell harboring the knock in has more functional protein active in the cell. As outlined herein, a knock in can be done in a variety of ways, usually by the introduction of at least one copy of a transgene (tg) encoding the protein into the cell, although this can also be done by replacing regulatory components as well, for example by adding a constitutive promoter to the endogeneous gene. In general, knock in technologies result in the integration of the extra copy of the transgene into the host cell.

The invention provides infectious gene therapy viruses, such as transgenic lentiviruses. They are prepared using engineered packaging cells that express Fc receptor proteins (e.g. CD16, CD32, CD64, or intracellular signalling domain truncations thereof). In order for such packaging cells to produce the gene therapy viruses, a transfer plasmid, one or two packaging plasmids, and one envelope plasmid are transfected into the cells. Such Fc receptor-expressing packaging cells (e.g. engineered HEK293t cells expressing CD64) produce gene therapy viruses expressing the Fc receptor proteins.

In some embodiments, the cell lines express a cell-surface Fc receptor that has been truncated to remove the intracellular signalling domain. Such intracellular signalling domain truncations may, for example, be a truncated CD16, CD32, or CD64 protein (CD16t, CD32t, or CD64t, respectively). These truncated proteins are represented by SEQ ID NOS: 11, 12, and 13. In other embodiments, the truncated proteins have at least a 90% sequence identity to SEQ ID NOS: 11, 12, and 13. In other embodiments, these truncated proteins are comprised within fusion proteins having heterogeneous transmembrane domains known in the art. See U.S. patent application Ser. No. 63/305,587 which is incorporated by reference herein in its entirety.

When these specialized packaging cells are transfected with the vector plasmid carrying the gene of interest and the above-mentioned packaging and envelope plasmids, the viral RNA is converted to proviral double-stranded DNA through a multistep process of reverse transcription. The proviral DNA then complexes with viral proteins to facilitate nuclear import and integration into the host genome. The process of integration is assisted by viral proteins. The integrated proviral genome relies on the host machinery to initiate and complete transcription and translation of viral proteins necessary to assemble infectious particles. The viral progeny then exits the cell through a process called budding in which virions are released into the extracellular space from the plasma membrane. During the budding process, the Fc receptor proteins present within the host cell membrane are incorporated into the viral envelopes.

Thus, the invention provides compositions and methodologies for generating packaging cells with enhanced Fc receptor (e.g. CD16, CD32, OR CD64) expression. In some aspects of the invention, the cells are derived from HEK293t cells or its derivatives (e.g. LentiPro26; Tomás, H. A., et al. Sci Rep 8:5271 (2018)), STAR cells (Ikeda, Y. et al. Nat. Biotechnol. 21:569-72 (2003)), or RD2-MolPack-Chim3 cells (Stornaiuolo, A. et al. Hum. Gene Ther. Methods 24:228-40 (2013)). The foregoing are incorporated by reference in their entirety.

The invention includes methods of modifying nucleic acid sequences within cells or in cell-free conditions to generate cells with enhanced CD16, CD32, or CD64 expression. Exemplary technologies include homologous recombination, knock-in, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

In some embodiments, the packaging cells of the invention include a reduction in MHC I function (HLA I when the cells are derived from human cells).

As will be appreciated by those in the art, the reduction in function can be accomplished in a number of ways, including removing nucleic acid sequences from a gene, interrupting the sequence with other sequences, or altering the regulatory components of the nucleic acid. For example, all or part of a coding region of the gene of interest can be removed or replaced with “nonsense” sequences, frameshift mutations can be made, all or part of a regulatory sequence such as a promoter can be removed or replaced, translation initiation sequences can be removed or replaced, etc.

As will be appreciated by those in the art, the successful reduction of the MHC I function (HLA I when the cells are derived from human cells) in the packaging cells can be measured using techniques known in the art and as described below; for example, flow cytometry techniques using labeled antibodies that bind the HLA complex; for example, using commercially available HLA-A,B,C antibodies that bind to the the alpha chain of the human major histocompatibility HLA Class I antigens.

In one embodiment, the reduction in HLA I proteins is done by disrupting the expression of the (3-2 microglobulin gene in the packaging cell, the human sequence of which is disclosed herein. This alteration is generally referred to herein as a gene “knockout,” and in the packaging cells of the invention it is done on both alleles in the host cell. Generally, the techniques to do both disruptions are the same.

A particularly useful embodiment uses CRISPR technology to disrupt the gene. In some cases, CRISPR technology is used to introduce small deletions/insertions into the coding region of the gene, such that no functional protein is produced, often the result of frameshift mutations that result in the generation of stop codons such that truncated, non-functional proteins are made.

Accordingly, a useful technique is to use CRISPR sequences designed to target the coding sequence of the B2m gene in mouse or the B2M gene in human. After gene editing, the transfected packaging cell cultures are dissociated to single cells. Single cells are expanded to full-size colonies and tested for CRISPR edit by screening for presence of aberrant sequence from the CRISPR cleavage site. Clones with deletions in both alleles are picked. Such clones do not express B2M as demonstrated by PCR and do not express HLA I as demonstrated by flow cytometry analysis.

Assays to test whether the B2M gene has been inactivated are known and described herein. In one embodiment, the assay is a Western blot of cell lysates probed with antibodies to the B2M protein. In another embodiment, reverse transcriptase polymerase chain reactions (rt-PCR) confirms the presence of the inactivating alteration.

In addition, the cells can be tested to confirm that the HLA I complex is not expressed on the cell surface. This may be assayed by flow cytometry analysis using antibodies to one or more HLA cell surface components as discussed above.

In addition to a reduction in HLA I, the packaging cells of the invention may also lack MHC II function (HLA II when the cells are derived from human cells).

As will be appreciated by those in the art, the reduction in function can be accomplished in a number of ways, including removing nucleic acid sequences from a gene, adding nucleic acid sequences to a gene, disrupting the reading frame, interrupting the sequence with other sequences, or altering the regulatory components of the nucleic acid. In one embodiment, all or part of a coding region of the gene of interest can be removed or replaced with “nonsense” sequences. In another embodiment, regulatory sequences such as a promoter can be removed or replaced, translation initiation sequences can be removed or replaced, etc.

The successful reduction of the MHC II function (HLA II when the cells are derived from human cells) in the pluripotent cells or their derivatives can be measured using techniques known in the art such as Western blotting using antibodies to the protein, FACS techniques, rt-PCR techniques, etc.

In one embodiment, the reduction in HLA-II proteins is done by disrupting the expression of the CIITA gene in the packaging cell, the human sequence of which is shown herein. This alteration is generally referred to herein as a gene “knock out,” and in the packaging cells of the invention it is done on both alleles in the host cell.

Assays to test whether the CIITA gene has been inactivated are known and described herein. In one embodiment, the assay is a Western blot of cell lysates probed with antibodies to the CIITA protein. In another embodiment, reverse transcriptase polymerase chain reactions (rt-PCR) confirms the presence of the inactivating alteration.

In addition, the cells can be tested to confirm that the HLA II complex is not expressed on the cell surface. Again, this assay is done as is known in the art and generally is done using either Western Blots or FACS analysis based on commercial antibodies that bind to human HLA Class II HLA-DR, DP and most DQ antigens as outlined below.

A particularly useful embodiment uses CRISPR technology to disrupt the CIITA gene. CRISPRs are designed to target the coding sequence of the CIITA gene, an essential transcription factor for all MHC II molecules. After gene editing, the transfected packaging cellcultures are dissociated into single cells. They are expanded to full-size colonies and tested for successful CRISPR editing by screening for the presence of an aberrant sequence from the CRISPR cleavage site. Clones with deletions do not express CIITA as determined by PCR and do not express MHC II/ HLA-II as determined by FACS analysis.

In addition to the reduction of HLA I and II (or MHC I and II), generally using B2M and CIITA knock-outs, the viruses produced in the packaging cells of the invention are less likely to induce an immune response by T cells, B cells, macrophages, and NK cell inactivation.

In some embodiments, reduced viral susceptibility to macrophage phagocytosis and NK cell inactivation results from increased CD47 on the packaging cell surface. This is done in several ways as will be appreciated by those in the art using “knock in” or transgenic technologies. In some cases, increased CD47 expression results from one or more CD47 transgenes.

Accordingly, in some embodiments, one or more copies of a CD47 gene is added to the packaging cells under control of an inducible or constitutive promoter, with the latter being preferred. In some embodiments, a lentiviral construct is employed as described herein or known in the art. CD47 genes may integrate into the genome of the host cell under the control of a suitable promoter as is known in the art.

The packaging cell lines can be generated from B2M−/− CIITA−/−0 packaging cells. To overexpress CD47, the CD47 transgene sequence may be synthesized and the DNA cloned into, e.g., the plasmid Lentivirus pLenti6/V5 with a blasticidin resistance (Thermo Fisher Scientific, Waltham, MA). Packaging cells expressing CD47 may be selected using a selectable markers such as Blasticidin. Packaging cells expressing CD47 can also be sorted using flow cytometry.

In some embodiments, the expression of the CD47 gene can be increased by altering the regulatory sequences of the endogenous CD47 gene, for example, by exchanging the endogenous promoter for a constitutive promoter or for a different inducible promoter. This can generally be done using known techniques such as CRISPR.

Once altered, the presence of sufficient CD47 expression can be assayed using known techniques such as Western blots, ELISA assays or flow cytometry assays using anti-CD47 antibodies. In general, “sufficiency” in this context means an increase in the expression of CD47 on the packaging cell surface that silences the inactivation of viral particles produced by this packaging cell by NK cell.

In some aspects of the invention, the packaging cells generated as discussed herein will be blood type O, Rh factor negative cells because the process will have started with packaging cells having an O− blood type.

In other embodiments of the invention, the packaging cells can be made blood type O by enzymatic conversion of A and B antigens. In preferred aspects, the B antigen is converted to O using an enzyme. In more preferred aspects, the enzyme is an α-galactosidase. This enzyme eliminates the terminal galactose residue of the B antigen. Other aspects of the invention involve the enzymatic conversion of A antigen to O. In preferred aspects, the A antigen is converted to O using an α-N-acetylgalactosaminidase. Enzymatic conversion is discussed, e.g., in Olsson et al., Transfusion Clinique et Biologique 11:33-39 (2004); U.S. Pat. Nos. 4,427,777, 5,606,042, 5,633,130, 5,731,426, 6,184,017, 4, 609,627, and 5,606,042; and Int'l Pub. No. W09923210, each of which are incorporated by reference herein in their entirety.

Other embodiments of the invention involve genetically modifying packaging cells to blood type O by engineering the cells to knock out Exon 7 of the ABO gene (NCBI Gene ID: 80908). Any knockout methodology known in the art or described herein, such as CRISPR, TALENs, Zn fingers, or homologous recombination, may be employed.

Other embodiments of the invention involve rendering the packaging cell Rh factor negative by knocking out the C and/or E antigens of the Rh blood group system (RH), K in the Kell system (KEL), Fya and Fy3 in the Duffy system (FY), Jkb in the Kidd system (JK), or U and/or S in the MNS blood group system. Alternatively, the cells of the invention may be made Rh negative by or silencing the SLC14A1 (JK) gene (NCBI Gene ID: 6563). Any knockout methodology known in the art or described herein, such as CRISPR, TALENs, Zn Fingers, or homologous recombination, may be employed.

As will be appreciated by those in the art, a number of different techniques can be used to engineer the cells of the invention. In general, these techniques can be used individually or in combination.

In one embodiment, the cells are manipulated using clustered regularly interspaced short palindromic repeats)/Cas (“CRISPR”) technologies as is known in the art. There are a large number of techniques based on CRISPR, see for example Doudna and Charpentier, Science doi:10.1126/science.1258096, hereby incorporated by reference. CRISPR techniques and kits are sold commercially.

In some embodiments, the packaging cells of the invention are made using Transcription Activator-Like Effector Nucleases (TALEN) methodologies. TALEN are restriction enzymes combined with a nuclease that can be engineered to bind to and cut practically any desired DNA sequence. TALEN kits are sold commercially.

In one embodiment, the cells are manipulated using Zn finger nuclease technologies. Zn finger nucleases are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms, similar to CRISPR and TALENs.

There are a wide variety of viral techniques that can be used to generate the packaging cells of the invention, including, but not limited to, the use of retroviral vectors, lentiviral vectors, adenovirus vectors, adeno-associated virus, and Sendai viral vectors. Lentiviruses can be used to effectively transduce packaging cells to express Fc receptors (e.g. CD16, CD32, or CD64). Episomal vectors used in the generation of iPSCs are described below.

Well-known recombinant techniques are used to generate recombinant nucleic acids as outlined herein. In certain embodiments, the recombinant nucleic acids (either than encode a desired polypeptide or disruption sequences) may be operably linked to one or more regulatory nucleotide sequences in an expression construct. Regulatory nucleotide sequences will generally be appropriate for the host cell and subject to be treated. Numerous types of appropriate expression vectors and suitable regulatory sequences are known in the art for a variety of host cells.

Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, the early promoter of human Cytomegalovirus (CMV), and the eukaryotic translation elongation factor 1 a (EF-1a) promoter. Examples of other heterologous mammalian promoters are the actin, immunoglobulin or heat shock promoter(s).

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a Hindlll E restriction fragment. Greenaway, P. J. et al., Gene 18: 355-360 (1982). The foregoing references are incorporated by reference in their entirety.

The gene therapy viruses of the invention have a reduced susceptibility to antibodies resulting from CD16, CD32, or CD64 in their viral envelopes. The resulting viruses sequester antibodies due to these membrane-bound proteins. In one embodiment, the viruses are expressed in packaging cells that comprise one or more CD16, CD32, or CD64 transgenes. This is done in several ways as will be appreciated by those in the art using “knock in” or transgenic technologies. In some cases, increased CD16, CD32, or CD64 expression results from one or more transgenes.

Accordingly, in some embodiments, one or more copies of a CD16, CD32, or CD64 gene is added to the cells under control of an inducible or constitutive promoter, with the latter being preferred. In some embodiments, a lentiviral construct is employed as described herein or known in the art. The genes may integrate into the genome of the host cell under the control of a suitable promoter as is known in the art.

Cells expressing Fc receptors (e.g. CD16, CD32, or CD64) are selected using a Blasticidin marker or another antibiotic resistance. The cells can also be selected via flow cytometry sorting. The gene sequence is synthesized and the DNA may be cloned, for instance, into the plasmid Lentivirus pLenti6N5 with a blasticidin resistance (Thermo Fisher Scientific, Waltham, MA)

In some embodiments, the expression of the gene can be increased by altering the regulatory sequences of the endogenous CD16, CD32, or CD64 gene, for example, by exchanging the endogenous promoter for a constitutive promoter or for a different inducible promoter. This can generally be done using known techniques such as CRISPR.

Once altered, the presence of sufficient expression can be assayed using known techniques such as those described in the Examples, such as Western blots, ELISA assays or flow cytometry assays using anti-CD16, CD32, or CD64 antibodies. In some embodiments, “sufficiency” in this context means an increase in expression at the cell surface that results in gene therapy viruses that sequester IgG antibodies and prevent opsonization.

The invention provides gene therapy viruses having increased Fc receptors on the viral envelope produced in the packaging cells disclosed herein. In some embodiments, the gene therapy viruses are used to treat or prevent diseases or disorders having a genetic basis. In some embodiments, the genetic disease is X-linked severe combined immunodeficiency (X-SCID), Stargardt Disease, Usher Syndrome, Choroideremia, Achromatopsia, X-linked Retinoschisis, P-thalassemia, Sickle Cell Disease, Hemophilia, Wiskott-Aldrich Syndrome, X-linked Chronic Granulomatos Disease, Mucopolysaccharidosis IIIB, Aromatic L-Amino Acid Decarboxylase Deficiency, Recessive Dystrophic Epidermolysis Bullosa, Mucopolysaccharidosis Type I (Hurler Syndrome), Alpha 1 Atnitrypsin Deficiency, Homozygous Familial Hypercholesterolemia, Hutchinson-Gilford Progeria Syndrome (HGPS), Acondroplasia, MECP2 Duplication Syndrome, Pendred Syndrome, Leber Hereditary Optic Neuropathy, Noonan Syndrome, Congenital Myasthenic Syndrome, or Hereditary Hemorhagic Telangiectasia.

Exemplary drug formulations of the invention include aqueous solutions, organic solutions, powder formulations, solid formulations and a mixed phase formulations.

Pharmaceutical compositions of this invention comprise any of the compounds of the present invention, and pharmaceutically acceptable salts thereof, with any pharmaceutically acceptable carrier, adjuvant or vehicle. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered by subcutaneous, transdermal, oral, parenteral, inhalation, ocular, topical, rectal, nasal, buccal (including sublingual), vaginal, or implanted reservoir modes. The pharmaceutical compositions of this invention may contain any conventional, non-toxic, pharmaceutically-acceptable carriers, adjuvants or vehicles. The term parenteral as used herein includes subcutaneous, intracutaneous, intravenous, intramuscular, intraarticular, intrasynovial, intrasternal, intrathecal, intralesional, and intracranial injection or infusion techniques.

Also contemplated, in some embodiments, are pharmaceutical compositions comprising as an active ingredient, therapeutic compounds described herein, or pharmaceutically acceptable salt thereof, in a mixture with a pharmaceutically acceptable, non-toxic component. As mentioned above, such compositions may be prepared for parenteral administration, particularly in the form of liquid solutions or suspensions; for oral or buccal administration, particularly in the form of tablets or capsules; for intranasal administration, particularly in the form of powders, nasal drops, evaporating solutions or aerosols; for inhalation, particularly in the form of liquid solutions or dry powders with excipients, defined broadly; for transdermal administration, particularly in the form of a skin patch or microneedle patch; and for rectal or vaginal administration, particularly in the form of a suppository.

The compositions may conveniently be administered in unit dosage form and may be prepared by any of the methods well-known in the pharmaceutical art, for example, as described in Remington's Pharmaceutical Sciences, 17th ed., Mack Publishing Co., Easton, PA (1985), incorporated herein by reference in its entirety. Formulations for parenteral administration may contain as excipients sterile water or saline alkylene glycols such as propylene glycol, polyalkylene glycols such as polyethylene glycol, saccharides, oils of vegetable origin, hydrogenated napthalenes, serum albumin or other nanoparticles (as used in Abraxane™, American Pharmaceutical Partners, Inc. Schaumburg, IL), and the like. For oral administration, the formulation can be enhanced by the addition of bile salts or acylcarnitines. Formulations for nasal administration may be solid or solutions in evaporating solvents such as hydrofluorocarbons, and may contain excipients for stabilization, for example, saccharides, surfactants, submicron anhydrous alpha-lactose or dextran, or may be aqueous or oily solutions for use in the form of nasal drops or metered spray. For buccal administration, typical excipients include sugars, calcium stearate, magnesium stearate, pregelatinated starch, and the like.

Delivery of modified therapeutic compounds described herein to a subject over prolonged periods of time, for example, for periods of one week to one year, may be accomplished by a single administration of a controlled release system containing sufficient active ingredient for the desired release period. Various controlled release systems, such as monolithic or reservoir-type microcapsules, depot implants, polymeric hydrogels, osmotic pumps, vesicles, micelles, liposomes, transdermal patches, iontophoretic devices and alternative injectable dosage forms may be utilized for this purpose. Localization at the site to which delivery of the active ingredient is desired is an additional feature of some controlled release devices, which may prove beneficial in the treatment of certain disorders.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Hely or a similar alcohol.

Dosage levels of between about 0.01 and about 100 mg/kg body weight per day, preferably 0.5 and about 50 mg/kg body weight per day of the active ingredient compound are useful in the prevention and treatment of disease. Such administration can be used as a chronic or acute therapy. The amount of drug that may be combined with the carrier to produce a single dosage form will vary depending upon the host treated and the particular mode of administration. A typical preparation will contain from about 5% to about 95% active compound (w/w). Preferably, such preparations contain from about 20% to about 80% active compound.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

As the skilled artisan will appreciate, lower or higher doses than those recited above may be required. Specific dosage and treatment regimens for any particular patient will depend upon a variety of factors, including the activity of the specific compound employed, the age, body weight, general health status, gender, diet, time of administration, rate of excretion, drug combination, the severity and course of an infection, the patient's disposition to the infection and the judgment of the treating physician.

In order that the invention described herein may be more fully understood, the following examples are set forth. It should be understood that these examples are for illustrative purposes only and are not to be construed as limiting this invention in any manner.

### EXAMPLES

### Example 1

### Generation of a CD64 HEK293T Packaging Cell Line

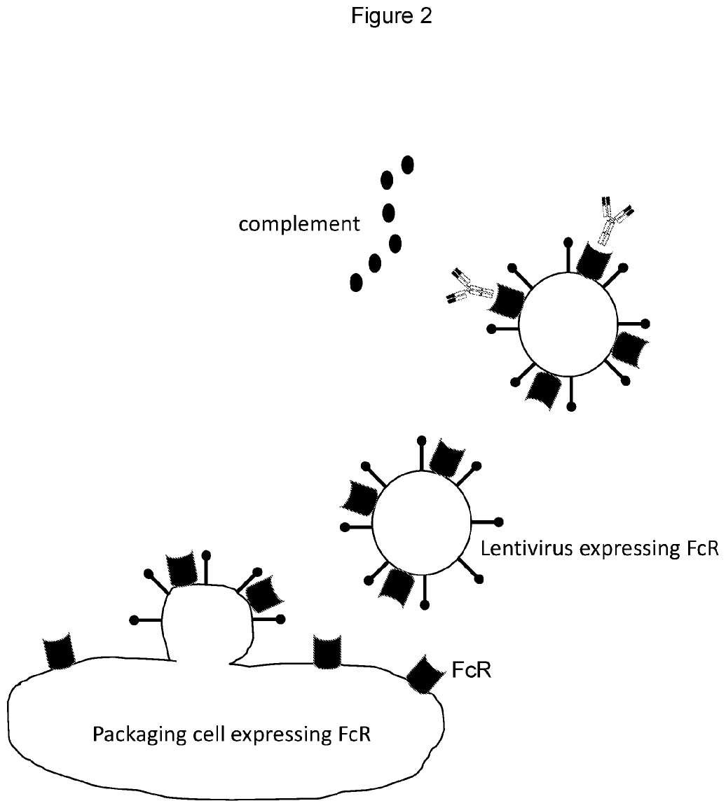

Gene editing is used to express Fc receptors (e.g. CD64, CD32, or CD16) on packaging cells. In this example, CD64 was expressed on 293t cells (FIG. 2).

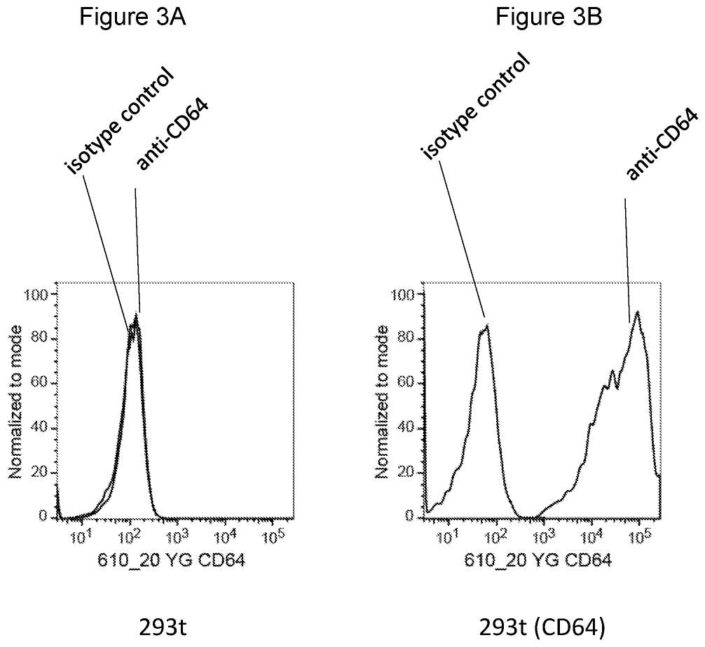

HEK293t (293t; ATCC CRL-3216) were purchased from ATCC (Manassas, VA) and grown in Dulbecco's Modified Eagle's Medium (DMEM; ATCC 30-2002) supplemented with 10% Fetal Bovine Serum (heat inactivated) (ATCC 30-2020), and 2mM L-glutamine (ATCC 30-2214). For transductions, 1.5×10^{5 293}t were plated per well in a 6-well plate. The cells were incubated overnight at 37° C. in a cell incubator. On the next day, one vial of CD64 lentiviral particles (Origene, Rockville, MD, CAT#: RC207487L2V) was added. After 24 h, some medium was added and after 48 h, medium was changed. Successfully transduced 293t (CD64) cells (compare FIGS. 3A and 3B) were enriched by flow cytometry sorting using an anti-CD64 antibody (CD64 PE: clone 10.1, BD).

### Example 2

### 293t (CD64) Adsorb Human IgG via Fc

The ability of the parental 293t cells and 293t (CD64) cells to bind human IgG antibody Fc was shown using flow cytometry. The cells were incubated with increasing concentrations of alemtuzumab (Bio-Rad, Hercules, CA, Catalog No. MCA6101), a humanized anti-CD52 antibody with a human IgG1 Fc domain. 293t do not express CD52. The binding of alemtuzumab Fc to the cells was detected using a goat anti-human IgG (heavy and light chains) secondary antibody (F(ab′)2) with Qdot 655 tag (Q-11221MP, Thermo Fisher Scientific, Carlsbad, CA).

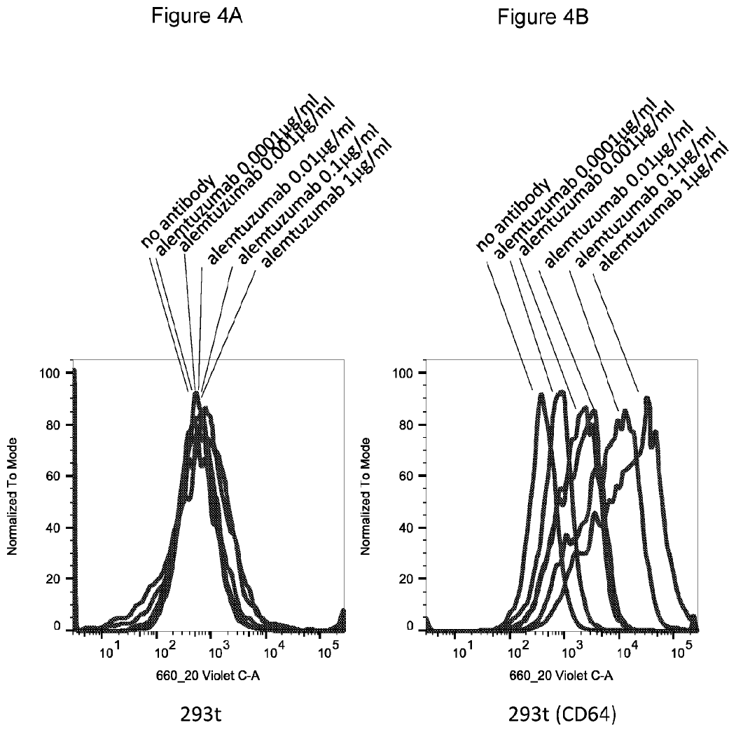

While unmodified 293t cells did not bind any alemtuzumab, 293t (CD64) cells showed increasing alemtuzumab binding with increasing antibody concentrations (FIGS. 4A and 4B). Specifically, FIG. 4A shows a flow cytometry histogram of alemtuzumab Fc binding to the parental 293t cells. The cells were exposed to increasing concentrations of alemtuzumab (IgG). There was no measurable IgG binding. FIG. 4B shows that 293t cells expressing CD64 captured and bound alemtuzumab in a concentration-dependent manner. The fact that the secondary antibody used only binds to the heavy and light chain domains and not Fc shows that the 293t cells bound alemtuzumab via the Fc domain.

### Example 3

### 293t (CD64) were Protected from Anti-HLA-A2 CDC

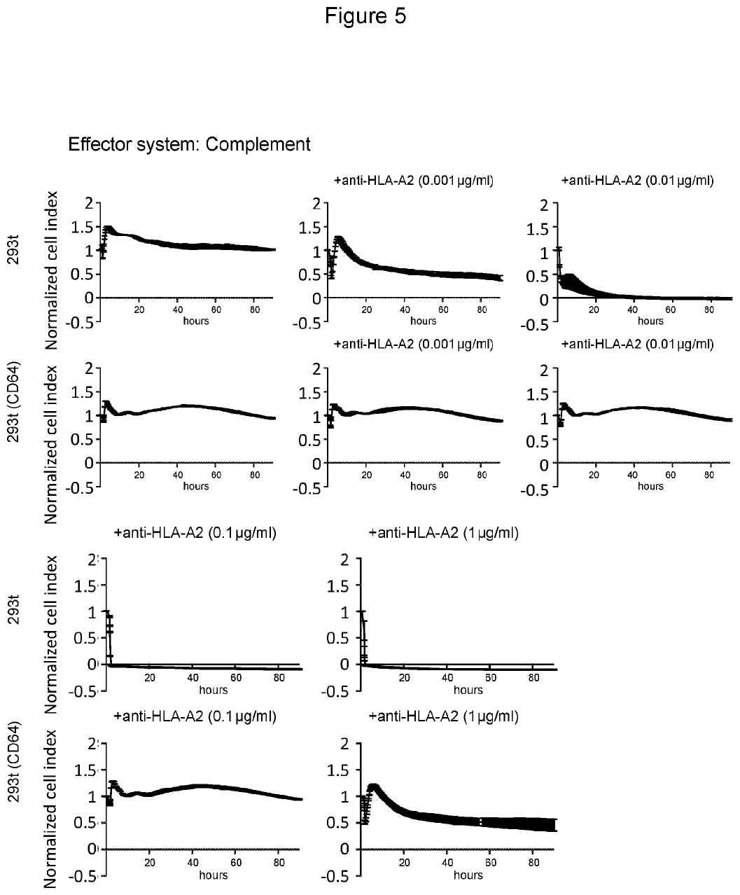

293t and 293t (CD64) cells were plated on the XCelligence platform for in vitro impedance assays (FIG. 5). They attached to plastic dishes and formed confluent layers. A recombinant, humanized monoclonal antibody to HLA-A2 (Ab00947-10.0, Absolute Antibody, Boston, MA) was added at a concentration of 0.001, 0.01, 0.1, or 1.0 μg/ml. Because 293t cells express HLA-A2, this antibody is capable of mediating CDC. Human serum was added to the wells to supply complement. Cell killing was assessed by measurig the impedance between electrodes on the special 96-well E-plates (ACEA BioSciences, San Diego, CA). A drop in impedance indicated a breakdown of the cell layer and shows target cell killing.

The upper row shows that increasing concentrations of anti-HLA-A2 led to increasing efficacy of 293t cell killing via CDC. The lower row shows that 293t (CD64) cells were protected against anti-HLA-A2 over several orders of magnitude.

### Example 4

### Gene Therapy Lentivirus Packaging in 293t and 293t (CD64) Cells

Both 293t and 293t (CD64) cells were then used to produce lentiviruses expressing firefly luciferase. The expression lentivector plasmid was co-transfected with lentiviral packaging plasmids (Cat#: HT-pack, Gentarget, San Diego, CA), into the packaging cell lines. Lentiviruses were packaged in DMEM medium with 10% serum. The crude lentiviruses were filtered by 0.45μm filter and concentrated to obtain the desired titer.

### Example 5

### Generation of a 293t-Fcgr1 Packaging Cell Line

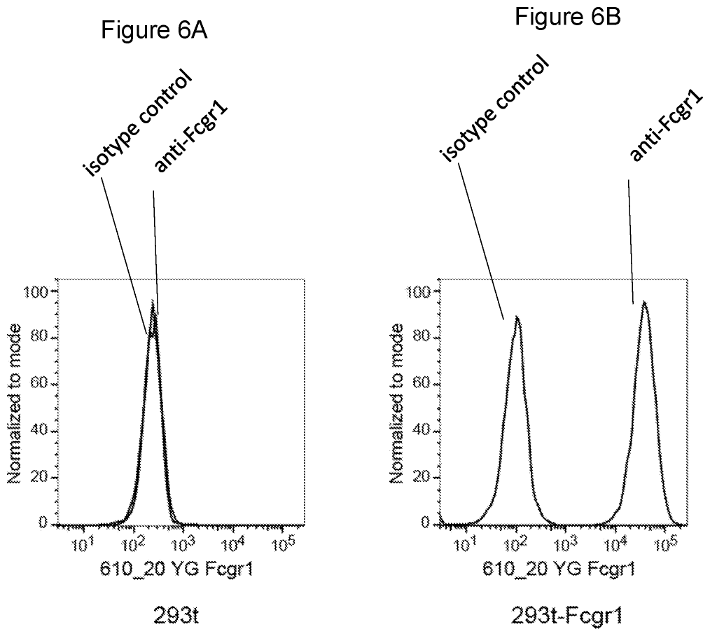

HEK293t (293t; ATCC CRL-3216) were purchased from ATCC (Manassas, VA) and grown in Dulbecco's Modified Eagle's Medium (DMEM; ATCC 30-2002) supplemented with 10% Fetal Bovine Serum (heat inactivated) (ATCC 30-2020), and 2 mM L-glutamine (ATCC 30-2214). For transductions, 1.5×10^{5 293}t were plated per well in a 6-well plate. The cells were incubated overnight at 37° C. in a cell incubator. On the next day, one vial of Fcgr1 lentiviral particles (custom order, GenTarget, San Diego) was added. After 24 h, some medium was added and after 48 h, medium was changed. Successfully transduced 293t-Fcgr1 cells (FIG. 6) were enriched by flow cytometry sorting using an anti-Fcgr1 antibody (PE: clone X54-5/7.1, Cat#: 139303, Biolegend) or mouse IgG1 isotype control (Cat#: 550617, Biolegend).

### Example 6

### 293t-Fcgr1 Adsorb Mouse IgG2a Via Fc

The ability of the parental 293t cells and 293t-Fcgr1 cells to bind mouse IgG2a antibody Fc was shown using flow cytometry. The cells were incubated with increasing concentrations of anti-CD20 (Cat#: ab219329, Abcam) mouse IgG2a. 293t cells do not express CD20. The binding of anti-CD20 Fc to the cells was detected using a goat anti-mouse IgG (heavy and light chains) secondary antibody (F(ab')2) with Qdot 655 tag (Q-11021MP, Thermo Fisher Scientific, Carlsbad, CA).

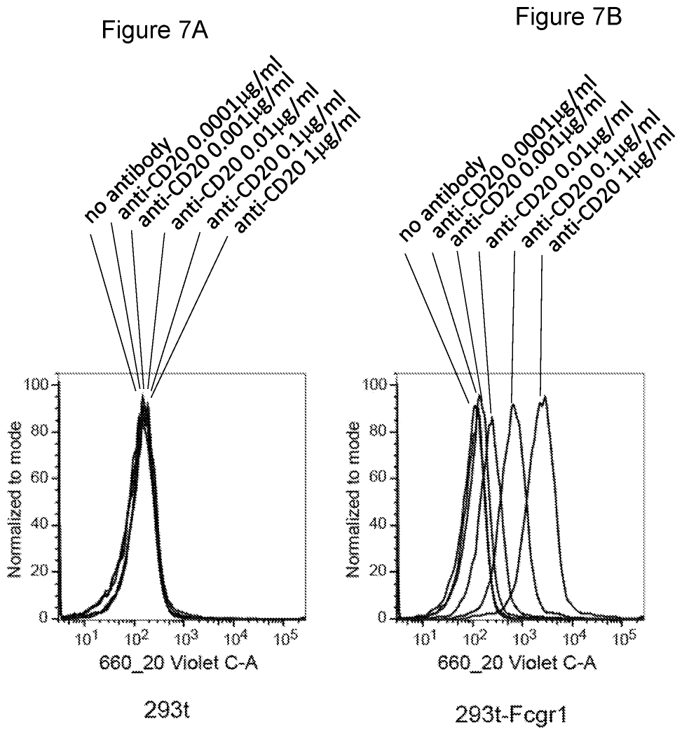

While unmodified 293t cells did not bind any anti-CD20, 293t-Fcgr1 cells showed increasing anti-CD20 binding with increasing antibody concentrations (FIGS. 7A and 7B). Specifically, FIG. 7A shows a flow cytometry histogram of anti-CD20 Fc binding to the parental 293t cells. The cells were exposed to increasing concentrations of alemtuzumab (IgG). There was no measurable IgG2a binding. FIG. 7B shows that 293t cells expressing Fcgr1 captured and bound anti-CD20 in a concentration-dependent manner. The fact that the secondary antibody used only binds to the heavy and light chain domains and not Fc shows that the 293t-Fcgr1 cells bound anti-CD20 via the Fc domain.

### Example 7

### Gene Therapy Lentivirus Packaging in 293t and 293t-Fcgr1 Cells

Both 293t and 293t-Fcgr1 cells were then used to produce lentiviruses expressing firefly luciferase. The expression lentivector plasmid was co-transfected with lentiviral packaging plasmids (Cat#: HT-pack, Gentarget, San Diego, CA), into the packaging cell lines. Lentiviruses were packaged in DMEM medium with 10% serum. The crude lentiviruses were filtered by 0.45 μm filter and concentrated to obtain the desired titer.

For ELISAs, concentrated VSV-G-pseudotyped 293t or 293t-Fcgr1 (>1×10e8 TU/mL) was used as the capture antigen. Nunc PVC 96 flat-well plates were coated (50 mL/well) with lentivirus diluted 1:1,000 (20 mg/mL) in 100 mM bicarbonate/carbonate binding buffer (3.03 g Na2CO3 and 6.0 g NaHCO3 in 1,000 mL distilled water, pH 9.6) and incubated overnight at 4 degrees C. The plates were washed three times with wash buffer (DPBS with 0.05% Tween-20; Sigma-Aldrich, St. Louis, MO, USA), followed by blocking with blocking buffer (DPBS with 1% fetal bovine serum [FBS]) for 1 h. A murine monoclonal anti-VSV-G antibody at 200 mg/mL (sc-365019, Santa Cruz Biotechnology, Santa Cruz, CA, USA) or a murine monoclonal anti-Fcgr1 (Biolegend; clone: X54-5/7.1, cat no. 139304) was diluted (25 μg/mL) in blocking buffer for a total of 100 mL/well. Alternatively, an anti-CD20 mouse IgG2a (ab219329, clone: rIGEL/773, Abcam) was used over a range of concentrations. The plate was incubated with gentle agitation at room temperature for 2 h and then washed 3 times with wash buffer. A secondary goat anti-mouse IgG-HRP antibodies (sc-2005, Santa Cruz Biotechnology, Santa Cruz, CA, USA) was used at 1:2,000 in blocking buffer and 50 mL was added to each well. After 1 h incubation with gentle agitation, the wells were washed 3 times with wash buffer. After aspiration of the last wash, 50 mL of HRP substrate (3,30,5,50-tetramethylbenzidine) liquid soluble HRP/chromatin/peroxidase substrate system (Moss, Pasadena, MD, USA) was added to each well. After a 5-min incubation at room temperature, 50 mL of HCl (0.1N) stop solution was added to each well. The absorbance (OD) was read at 450 nm and background subtracted out.

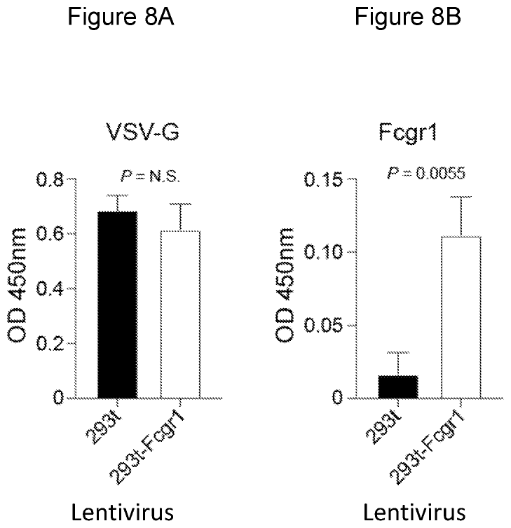

FIG. 8A shows that both lentiviruses 293t and 293t-Fcgr1 had similar VSV-G pseudotyping. However, Fcgr1 was largely increased on 293t-Fcgr1 lentiviruses (FIG. 8B).

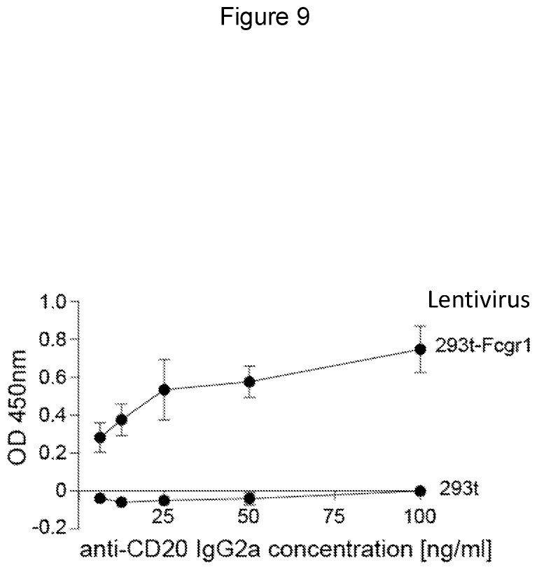

FIG. 9 shows the binding of anti-CD20 IgG2a via Fc to lentiviruses 293t and 293t-Fcgr1. We did not detect any antibody binding to lentivirus 293t, but we found a concentration-dependent antibody binding to 293t-Fcgr1.

### Example 8

### Gene Therapy Lentivirus from 293t-Fcgr1 Cells are Protected from CDI

A total of 1×10⁴ mouse endothelial cells were plated in 96 wells and let attach for 24 h. Then, 2 μl of lentivirus 293t or lentivirus 293t-Fcgr1 (1×10e8 IFU/ml) was added to the cells with mouse serum to supplement complement. Also, anti-VSV-G mouse IgG2a was added in a range of concentrations. Components were mixed with 8 μg/ml Polybrene in total volume of 150 μl/well to facilitate transduction. After 2 days, the bioluminescence signal was quantified. All experiments were done in triplicates.

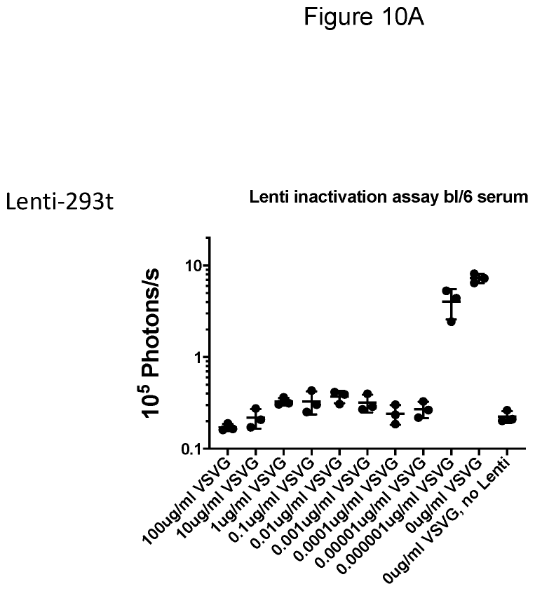

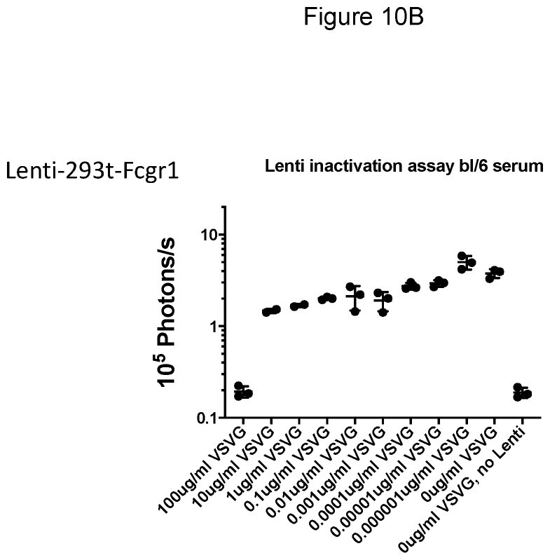

FIG. 10A shows good transduction efficiency of the lentivirus 293t when no anti-VSV-G was added. However, anti-VSV-G led to concentration-dependent inactivation of the lentiviral particles with low bioluminescence signals. In contrast, FIG. 10B show that lentiviruses 293t-Fcgr1 were mostly resistant against anti-VSV-G IgG2a in mouse serum.

### Example 9

### Gene Therapy Lentivirus from 293t (CD64) Cells are Protected from CDI

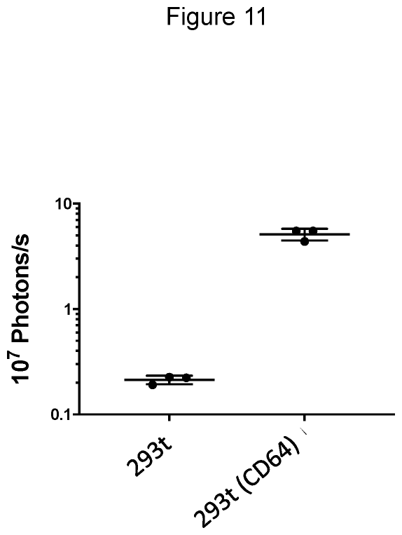

An in vitro lentivirus inactivation assay is performed. BALB/c mice are intravenously injected with 30 million lentiviral particles packaged in 293t cells and 28 days later, serum containing anti-lentiviral antibodies is drawn. Target human endothelial cells are plated in 96 wells coated with gelatine and allowed to attach overnight. The next day, different doses of lentivirus packaged in 293t or 293t (CD64) is incubated with serum containing anti-lentiviral antibodies. Then the serum/lentivirus mix is added to the endothelial cells and 2 days later, the transduction efficacy is measured using BLI imaging. The lentivirus packaged in 293t (CD64) evades the antibody inactivation and achieve higher BLI signals (FIG. 11).

### I. Exemplary Sequences:

Human CD64
NM_000566
SEQ ID NO: 1
MWFLTTLLLWVPVDGQVDTTKAVITLQPPWVSVFQEETVT
LHCEVLHLPGSSSTQWFLNGTATQTSTPSYRITSASVNDS
GEYRCQRGLSGRSDPIQLEIHRGWLLLQVSSRVFTEGEPL
ALRCHAWKDKLVYNVLYYRNGKAFKFFHWNSNLTILKTNI
SHNGTYHCSGMGKHRYTSAGISVTVKELFPAPVLNASVTS
PLLEGNLVTLSCETKLLLQRPGLQLYFSFYMGSKTLRGRN
TSSEYQILTARREDSGLYWCEAATEDGNVLKRSPELELQV
LGLQLPTPVWFHVLFYLAVGIMFLVNTVLWVTIRKELKRK
KKWDLEISLDSGHEKKVISSLQEDRHLEEELKCQEQKEEQ
LQEGVHRKEPQGAT
Human CD52
NM_001803
SEQ ID NO: 2
MKRFLFLLLTISLLVMVQIQTGLSGQNDTSQTSSPSASSN
ISGGIFLFFVANAIIHLFCES
Human CD16 FCGR3A
NM_001803
SEQ ID NO: 3
MAEGTLWQILCVSSDAQPQTFEGVKGADPPTLPPGSFLPG
PVLWWGSLARLQTEKSDEVSRKGNWWVTEMGGGAGERLFT
SSCLVGLVPLGLRISLVTCPLQCGIMWQLLLPTALLLLVS
AGMRTEDLPKAVVFLEPQWYRVLEKDSVTLKCQGAYSPED
NSTQWFHNESLISSQASSYFIDAATVDDSGEYRCQTNLST
LSDPVQLEVHIGWLLLQAPRWVFKEEDPIHLRCHSWKNTA
LHKVTYLQNGKGRKYFHHNSDFYIPKATLKDSGSYFCRGL
FGSKNVSSETVNITITQGLAVSTISSFFPPGYQVSFCLVM
VLLFAVDTGLYFSVKTNIRSSTRDWKDHKFKWRKDPQDK
Human CD16 FCGR3B
NM_001803
SEQ ID NO: 4
MWQLLLPTALLLLVSAGMRTEDLPKAVVELEPQWYSVLEK
DSVTLKCQGAYSPEDNSTQWFHNENLISSQASSYFIDAAT
VNDSGEYRCQTNLSTLSDPVQLEVHIGWLLLQAPRWVFKE
EDPIHLRCHSWKNTALHKVTYLQNGKDRKYFHHNSDFHIP
KATLKDSGSYFCRGLVGSKNVSSETVNITITQGLAVSTIS
SFSPPGYQVSFCLVMVLLFAVDTGLYFSVKTNI
Human CD32 FCGR2A
NM_001803
SEQ ID NO: 5
MTMETQMSQNVCPRNLWLLQPLTVLLLLASADSQAAAPPK
AVLKLEPPWINVLQEDSVTLTCQGARSPESDSIQWFHNGN
LIPTHTQPSYRFKANNNDSGEYTCQTGQTSLSDPVHLTVL
SEWLVLQTPHLEFQEGETIMLRCHSWKDKPLVKVTFFQNG
KSQKESHLDPTFSIPQANHSHSGDYHCTGNIGYTLFSSKP
VTITVQVPSMGSSSPMGIIVAVVIATAVAAIVAAVVALIY
CRKKRISANSTDPVKAAQFEPPGRQMIAIRKRQLEETNND
YETADGGYMTLNPRAPTDDDKNIYLTLPPNDHVNSNN
Human CD32 FCGR2B
NM_001803
SEQ ID NO: 6
MGILSFLPVLATESDWADCKSPQPWGHMLLWTAVLFLAPV
AGTPAAPPKAVLKLEPQWINVLQEDSVTLTCRGTHSPESD
SIQWFHNGNLIPTHTQPSYRFKANNNDSGEYTCQTGQTSL
SDPVHLTVLSEWLVLQTPHLEFQEGETIVLRCHSWKDKPL
VKVTFFQNGKSKKFSRSDPNESIPQANHSHSGDYHCTGNI
GYTLYSSKPVTITVQAPSSSPMGIIVAVVTGIAVAAIVAA
VVALIYCRKKRISALPGYPECREMGETLPEKPANPTNPDE
ADKVGAENTITYSLLMHPDALEEPDDQNRI
Human CD32 FCGR2C
NM_001803
SEQ ID NO: 7
MGILSFLPVLATESDWADCKSPQPWGHMLLWTAVLFLAPV
AGTPAAPPKAVLKLEPQWINVLQEDSVTLTCRGTHSPESD
SIPWFHNGNLIPTHTQPSYRFKANNNDSGEYTCQTGQTSL
SDPVHLTVLSEWLVLQTPHLEFQEGETIVLRCHSWKDKPL
VKVTFFQNGKSKKESRSDPNFSIPQANHSHSGDYHCTGNI
GYTLYSSKPVTITVQAPSSSPMGIIVAVVTGIAVAAIVAA
VVALIYCRKKRISANSTDPVKAAQFEPPGRQMIAIRKRQP
EETNNDYETADGGYMTLNPRAPTDDDKNIYLTLPPNDHVN
SNN
Human β-2-Microglobulin
SEQ ID NO: 8
MSRSVALAVLALLSLSGLEAIQRTPKIQVYSRHPAENGKS
NFLNCYVSGFHPSDIEVDLLKNGERIEKVEHSDLSFSKDW
SFYLLYYTEFTPTEKDEYACRVNHVTLSQPKIVKWDRDI
Human CIITA protein, 160 amino acid
N-terminus
SEQ ID NO: 9
MRCLAPRPAGSYLSEPQGSSQCATMELGPLEGGYLELLNS
DADPLCLYHFYDQMDLAGEEEIELYSEPDTDTINCDQFSR
LLCDMEGDEETREAYANIAELDQYVFQDSQLEGLSKDIFK
HIGPDEVIGESMEMPAEVGQKSQKRPFPEELPADLKHWKP
Human CD47
SEQ ID NO: 10
MWPLVAALLLGSACCGSAQLLFNKTKSVEFTFCNDTVVIP
CFVTNMEAQNTTEVYVKWKFKGRDIYTFDGALNKSTVPTD
FSSAKIEVSQLLKGDASLKMDKSDAVSHTGNYTCEVTELT
REGETIIELKYRVVSWFSPNENILIVIFPIFAILLFWGQF
GIKTLKYRSGGMDEKTIALLVAGLVITVIVIVGAILFVPG
EYSLKNATGLGLIVTSTGILILLHYYVFSTAIGLTSFVIA
ILVIQVIAYILAVVGLSLCIAACIPMHGPLLISGLSILAL
AQLLGLVYMKFVE
Truncated human CD16
SEQ ID NO: 11
MWQLLLPTALLLLVSAGMRTEDLPKAVVFLEPQWYRVLEK
DSVTLKCQGAYSPEDNSTQWFHNESLISSQASSYFIDAAT
VDDSGEYRCQTNLSTLSDPVQLEVHIGWLLLQAPRWVFKE
EDPIHLRCHSWKNTALHKVTYLQNGKGRKYFHHNSDFYIP
KATLKDSGSYFCRGLFGSKNVSSETVNITITQGLAVSTIS
SFFPPGYQVSFCLVMVLLFAVDTGLYFSV
Truncated human CD32
SEQ ID NO: 12
MTMETQMSQNVCPRNLWLLQPLTVLLLLASADSQAAAPPK
AVLKLEPPWINVLQEDSVTLTCQGARSPESDSIQWFHNGN
LIPTHTQPSYRFKANNNDSGEYTCQTGQTSLSDPVHLTVL
SEWLVLQTPHLEFQEGETIMLRCHSWKDKPLVKVTFFQNG
KSQKESHLDPTFSIPQANHSHSGDYHCTGNIGYTLFSSKP
VTITVQVPSMGSSSPMGIIVAVVIATAVAAIVAAVVALIY
Truncated human CD64
SEQ ID NO: 13
MWFLTTLLLWVPVDGQVDTTKAVITLQPPWVSVFQEETVT
LHCEVLHLPGSSSTQWELNGTATQTSTPSYRITSASVNDS
GEYRCQRGLSGRSDPIQLEIHRGWLLLQVSSRVFTEGEPL
ALRCHAWKDKLVYNVLYYRNGKAFKFFHWNSNLTILKTNI
SHNGTYHCSGMGKHRYTSAGISVTVKELFPAPVLNASVTS
PLLEGNLVTLSCETKLLLQRPGLQLYFSFYMGSKTLRGRN
TSSEYQILTARREDSGLYWCEAATEDGNVLKRSPELELQV
LGLQLPTPVWFHVLFYLAVGIMFLVNTVLWVTI
Murine Fcgr1
SEQ ID NO: 14
MILTSFGDDMWLLTTLLLWVPVGGEVVNATKAVITLQPPW
VSIFQKENVTLWCEGPHLPGDSSTQWFINGTAVQISTPSY
SIPEASFQDSGEYRCQIGSSMPSDPVQLQIHNDWLLLQAS
RRVLTEGEPLALRCHGWKNKLVYNVVFYRNGKSFQFSSDS
EVAILKTNLSHSGIYHCSGTGRHRYTSAGVSITVKELFTT
PVLRASVSSPFPEGSLVTLNCETNLLLQRPGLQLHESFYV
GSKILEYRNTSSEYHIARAEREDAGFYWCEVATEDSSVLK
RSPELELQVLGPQSSAPVWFHILFYLSVGIMFSLNTVLYV
KIHRLQREKKYNLEVPLVSEQGKKANSFQQVRSDGVYEEV
TATASQTTPKEAPDGPRSSVGDCGPEQPEPLPPSDSTGAQ
TSQS*

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

What is claimed:

1. A gene therapy virus, comprising a transgenic genetic element and a viral envelop having a membrane-bound Fc Receptor protein.

2. The gene therapy virus of claim 1, wherein said membrane-bound protein is CD16, CD32, or CD64.

3. The gene therapy virus of claim 2, wherein said membrane-bound protein is CD64.

4. The gene therapy virus of claim 2, wherein said membrane-bound protein is CD32.

5. The gene therapy virus of any one of claims 1-4, wherein said virus is a lentivirus, retrovirus, or a sendai virus.

6. A modified packaging cell, wherein said modified cell has an elevated level of an Fc receptor protein expression when compared to a parental version of said modified packaging cell, wherein a first gene therapy virus produced in said modified packaging cell resists opsonization, complement-dependent inactivation (CDI) or antibody-dependent inactivation (ADI) in a subject when compared to a second gene therapy virus produced in said parental version of said modified packaging cell.

7. The modified packaging cell of claim 6, wherein said elevated Fc receptor protein is CD16, CD32, CD64, or truncated CD64 (CD64t).

8. The modified packaging cell of claim 7, wherein said elevated Fc receptor protein is CD64 and wherein said CD64 protein has at least a 90% sequence identity to SEQ ID NO:1.

9. The modified packaging cell of claim 8, wherein said CD64 protein has the sequence of SEQ ID NO:1

10. The modified packaging cell of any one of claims 6-9, wherein said modified packaging cell is from a species that is selected from the group consisting of a human, monkey, cow, pig, chicken, mouse, rat, hamster, guinea pig, and insect.

11. The modified packaging cell of any one of claims 6-9, wherein said modified packaging cell is derived from a human HEK293T cell, a LentiPro26 cell, a STAR cell, or an RD2-MolPack-Chim3 cell.

12. The modified packaging cell of claim 11, wherein said modified packaging cell is derived from a HEK293T cell.

13. The modified packaging cell of claim 7, wherein said increased CD16, CD32, CD64, or truncated CD64 (CD64t) expression results from introducing at least one copy of a human CD16, CD32, CD64, or truncated CD64 (CD64t) gene under the control of a promoter into said parental version of said modified pluripotent cell.

14. The modified packaging cell of claim 13, wherein said promoter is a constitutive promoter.

15. The modified packaging cell of any one of claims 6-14, further comprising a reduced HLA I expression level.

16. The modified packaging cell or cell derived therefrom of claim 15, wherein said HLA I function is reduced by a reduction in expression of a13-2 microglobulin, HLA-A, HLA-B, or HLA-C protein.

17. The modified packaging cell of claim 16, wherein said 13-2 microglobulin, HLA-A, HLA-B, or HLA-C protein expression is eliminated.

18. The modified packaging cell or cell derived therefrom of of claim 17, wherein a gene encoding said β-2 microglobulin, HLA-A, HLA-B, or HLA-C protein is knocked out.

19. The modified packaging cell of any one of claims 6-18, further comprising a reduced HLA II expression level.

20. The modified packaging cell or cell derived therefrom of claim 19, wherein said HLA II function is reduced by a reduction in expression of a RFXS, RFXANK, RFXAP, or CIITA, HLA-DP, HLA-DR, or HLA-DQ protein.

21. The modified packaging cell of claim 20, wherein said RFXS, RFXANK, RFXAP, or CIITA, HLA-DP, HLA-DR, or HLA-DQ protein expression is eliminated.

22. The modified packaging cell or cell derived therefrom of of claim 20, wherein a gene encoding said RFXS, RFXANK, RFXAP, or CIITA, HLA-DP, HLA-DR, or an HLA-DQ protein is knocked out.

23. The modified packaging cell of any one of claims 6-22, further comprising an increased CD47 expression when compared to a parental packaing cell.

24. The modified packaging cell of claim 23, wherein said increased CD47 protein expression results from a modification to an endogenous CD47 gene locus.

25. The modified packaging cell of claim 23, wherein said increased CD47 protein expression results from a CD47 transgene.

26. The modified packaging cell of any one of claims 6-25, wherein the cell is ABO blood group type 0.

27. The modified packaging cell of any one of claims 6-26, wherein said cell has a reduced or eliminated ABO blood group antigen selected from the group consisting of A1, A2, and B.

28. The modified packaging cell of any one of claims 6-27, wherein said cell is Rhesus factor negative (Rh−).

29. The modified packaging cell of any one of claims 6-28, wherein said cell has a reduced or eliminated Rh protein antigen expression selected from the group consisting of Rh C antigen, Rh E antigen, Kell K antigen (KEL), Duffy (FY) Fya antigen, Duffy Fy3 antigen, Kidd (JK) Jkb antigen, MNS antigen U, and MNS antigen S.

30. A gene therapy virus comprising a therapeutic transgenic genetic element and a viral envelope, wherein said gene therapy virus is produced from the modified packaging cell of any one of claims 6-29.

31. The gene therapy virus of any one of claim 1-5 or 30, wherein said viral envelop has no HLA I proteins, no HLA I proteins, or comprises CD47 proteins.

32. The gene therapy virus of any one of claim 1-5 or 30-31, wherein said gene therapy virus has an ABO blood type O phenotype or an Rh(−) phenotype.

33. A pharmaceutical composition for treating a disease in a subject, comprising the gene therapy virus of any one of claim 1-5 or 30-32 and a pharmaceutically acceptable carrier.

34. A medicament, comprising the gene therapy virus of any one of claim 1-5 or 30-32 and a pharmaceutically acceptable carrier.

35. The gene therapy virus of any one of claim 1-5 or 30-32 for treating a disease in a subject.

36. A method of manufacturing a pharmaceutical composition, comprising combining the gene therapy virus of any one of claim 1-5 or 30-32 with a pharmaceutical carrier.

37. A method of treating a genetic disease, comprising administering the gene therapy virus of any one of claim 1-5 or 30-32 to a subject.

38. A use of the gene therapy virus of any one of claim 1-5 or 20-32 for preparing a therapeutic composition for treating a genetic disease.

39. A use of the gene therapy virus of any one of claim 1-5 or 20-32 for treating a genetic disease.

40. A medicament for treating a genetic disease comprising the gene therapy virus of any one of claim 1-5 or 20-32.

41. The method of claims 37, wherein said genetic disease is selected from the group consisting of X-linked severe combined immunodeficiency (X-SCID), Stargardt Disease, Usher Syndrome, Choroideremia, Achromatopsia, X-linked Retinoschisis, P-thalassemia, Sickle Cell Disease, Hemophilia, Wiskott-Aldrich Syndrome, X-linked Chronic Granulomatos Disease, Mucopolysaccharidosis IIIB, Aromatic L-Amino Acid Decarboxylase Deficiency, Recessive Dystrophic Epidermolysis Bullosa, Mucopolysaccharidosis Type I (Hurler Syndrome), Alpha 1 Atnitrypsin Deficiency, Homozygous Familial Hypercholesterolemia, Hutchinson-Gilford Progeria Syndrome (HGPS), Acondroplasia, MECP2 Duplication Syndrome, Pendred Syndrome, Leber Hereditary Optic Neuropathy, Noonan Syndrome, Congenital Myasthenic Syndrome, and Hereditary Hemorhagic Telangiectasia.
